(19) United States
(12) Patent Application Publication
(10) Pub. No.: US 2022/0402874 A1
(43) Pub. Date: Dec. 22, 2022

(54) SOLID FORMS OF OMECAMTIV MECARBIL DIHYDROCHLORIDE AND PROCESSES THEREOF

(71) Applicant: DR. REDDY'S LABORATORIES LIMITED, Hyderabad, Telangana (IN)

(72) Inventors: Saikat Sen, West Bengal (IN); Srinivas Oruganti, Hyderabad (IN); Deepika Pathivada, Andhra Pradesh (IN); Satish Chowdary Nekkanti, Hyderabad (IN)

(73) Assignee: DR. REDDY'S LABORATORIES LIMITED, Hyderabad, Telangana (IN)

(21) Appl. No.: 17/767,278

(22) PCT Filed: Oct. 9, 2020
(86) PCT No.: PCT/IB2020/059485

§ 371 Date: Apr. 7, 2022

(30) Foreign Application Priority Data
Oct. 9, 2019 (IN) 201941040735
Dec. 30, 2019 (IN) 201941054410
Feb. 17, 2020 (IN) 202041006720
Feb. 27, 2020 (IN) 202041008254
Mar. 19, 2020 (IN) 202041011896
Mar. 31, 2020 (IN) 202041014393

(51) Int. Cl. C07D 213/75; A61K 9/19; A61K 9/16

(52) U.S. Cl. CPC C07D 213/75; A61K 9/19; A61K 9/1635; A61K 9/1611; A61K 9/1641; C07B 2200/13

(57) ABSTRACT

Aspects of the present application relate to solid forms of Omecamtiv mecarbil dihydrochloride. Specific aspects relate to crystalline forms and amorphous solid dispersions of Omecamtiv mecarbil dihydrochloride. Further specific aspects related to crystalline forms DP1, DP2, DP3, DP4, OMT-D, OMT-L, OMT-O, OMT-F, OMT-FS and amorphous solid dispersions of Omecamtiv mecarbil dihydrochloride and pharmaceutical compositions thereof.

### INTRODUCTION

Aspects of the present application relate to solid forms of Omecamtiv mecarbil dihydrochloride. Specific aspects relate to crystalline forms and amorphous solid dispersions of Omecamtiv mecarbil dihydrochloride.

Omecamtiv mecarbil is a first-in-class cardiac myosin activator, which increases the proportion of myosin heads that are tightly bound to actin and creates a force-producing state that is not associated with cytosolic calcium accumulation. Omecamtiv mecarbil is developed by Cytokinetics Inc. & Licensee, Amgen Inc. and has a chemical name: Methyl4-[(2-fluoro-3-{[(6-methyl (3-pyridyl))amino]carbonylamino}phenyl) methyl]piperazine carboxylate and the structure as below.

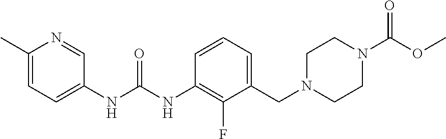

WO 2006009726 A2 (the '726 application) first discloses Omecamtiv mecarbil, its preparative process, pharmaceutical composition and its use in treating heart failure.

WO 2014152270 A1 (the '270 application) describes the dihydrochloride of Omecamtiv mecarbil exists as a crystalline hydrate Form A characterized by an X-ray powder diffraction pattern comprising peaks at about 6.6, 14.9, 20.1, 21.4, and 26.8±0.2° 2θ using Cu Kα radiation and anhydrous forms B and C, which are metastable.

WO 2020037164 A1 (the '164 application) discloses the crystalline form III to VII of Omecamtiv mecarbil free base, various other salts of Omecamtive mecarbil free base and polymorphs thereof.

WO 2020014406 A1 (the '406 application) discloses crystalline forms of Omecamtiv mecarbil dihydrochloride with acids including glycolic acid, citric acid, malic acid and malonic acid.

Further, Org. Process Res. Dev. 2015, 19, 12, 1842-1848 discloses PXRD patterns of anhydrous and hydrate forms of Omecamtiv mecarbil free base.

However, there remains a need for alternate solid forms of Omecamtiv mecarbil dihydrochloride and preparative processes thereof, exhibiting desired properties such as bioavailability and stability. Hence, it is desirable to provide a viable solid form of Omecamtiv mecarbil dihydrochloride.

### SUMMARY

In another aspect, the present application provides an amorphous solid dispersion of Omecamtiv mecarbil or its hydrochloride salt together with at least one pharmaceutically acceptable excipient.

In another aspect, the present application provides a stable amorphous premix comprising an amorphous solid dispersion of Omecamtiv mecarbil or its hydrochloride salt together with at least one pharmaceutically acceptable excipient and Syloid.

In another aspect, the present application provides a crystalline Form DP1 of Omecamtiv mecarbil dihydrochloride, characterized by a PXRD pattern comprising the peaks at about 6.55, 16.69, 25.53 and 26.42±0.2° 2θ.

In another aspect, the present application provides a crystalline Form DP2 of Omecamtiv mecarbil dihydrochloride, characterized by a PXRD pattern comprising the peaks at about 5.89, 17.94, 25.58 and 30.88±0.2° 2θ.

In another aspect, the present application provides a crystalline Form DP3 of Omecamtiv mecarbil dihydrochloride, characterized by a PXRD pattern comprising the peaks at about 25.64, 13.93 and 31.17±0.2° 2θ.

In another aspect, the present application provides a crystalline Form DP4 of Omecamtiv mecarbil dihydrochloride, characterized by a PXRD pattern comprising the peaks at about 6.8, 12.62 and 18.54±0.2° 2θ.

In another aspect, the present application provides a crystalline Form OMT-L of Omecamtiv mecarbil dihydrochloride, characterized by a PXRD pattern comprising the peaks at about 5.87, 17.84, 23.8, 25.35 and 27.91±0.2° 2θ.

In another aspect, the present application provides a crystalline Form OMT-D of Omecamtiv mecarbil dihydrochloride, characterized by a PXRD pattern comprising the peaks at about 12.98, 15.79, 18.55 and 25.63±0.2° 2θ.

In another aspect, the present application provides a crystalline Form OMT-O of Omecamtiv mecarbil dihydrochloride and oxalic acid, characterized by a PXRD pattern comprising the peaks at about 15.69, 24.26 and 29.44±0.2° 2θ.

In another aspect, the present application provides a crystalline Form OMT-F of Omecamtiv mecarbil dihydrochloride and fumaric acid, characterized by a PXRD pattern comprising the peaks at about 6.14 and 17.37°±0.2° 2θ.

In another aspect, the present application provides a crystalline Form OMT-FS of Omecamtiv mecarbil dihydrochloride characterized by a PXRD pattern comprising the peaks at about 24.68, 25.61 and 31.06°±0.2° 2θ.

In another aspect, the present application provides a process for the preparation of amorphous solid dispersion of Omecamtiv mecarbil or its hydrochloride salt, comprising the steps of providing a solution of Omecamtiv mecarbil or its hydrochloride salt together with at least one pharmaceutically acceptable excipient in a solvent and removing the solvent.

In another aspect, the present application provides a process for the preparation of crystalline Omecamtiv mecarbil dihydrochloride, comprising a step of contacting the solution of Omecamtiv mecarbil dihydrochloride in solvent with an anti-solvent.

In another aspect, the present application provides a process for the preparation of crystalline form DP1 of Omecamtiv mecarbil dihydrochloride, comprising the step of contacting Omecamtiv mecarbil dihydrochloride with formic acid.

In another aspect, the present application provides a process for the preparation of crystalline form DP2 of Omecamtiv mecarbil dihydrochloride, comprising the step of contacting Omecamtiv mecarbil dihydrochloride with acetic acid.

In another aspect, the present application provides a process for the preparation of crystalline form DP3 of Omecamtiv mecarbil dihydrochloride, comprising the step of heating crystalline form DP2 of Omecamtiv mecarbil dihydrochloride.

In another aspect, the present application provides a process for the preparation of crystalline form DP4 of Omecamtiv mecarbil dihydrochloride, comprising the steps of
a) contacting Omecamtiv mecarbil dihydrochloride with propionic acid
b) isolating Omecamtiv mecarbil dihydrochloride; and
c) optionally, heating crystalline Omecamtiv mecarbil dihydrochloride of step b).

In another aspect, the present application provides a process for the preparation of crystalline form OMT-L of Omecamtiv mecarbil dihydrochloride, comprising the steps of
a) contacting Omecamtiv mecarbil dihydrochloride with DL-Lactic acid
b) isolating Omecamtiv mecarbil dihydrochloride; and
c) optionally, heating crystalline Omecamtiv mecarbil dihydrochloride of step b).

In another aspect, the present application provides a process for the preparation of crystalline form OMT-D of Omecamtiv mecarbil dihydrochloride, comprising the steps of
a) contacting Omecamtiv mecarbil dihydrochloride with dimethyl sulfoxide;
b) isolating Omecamtiv mecarbil dihydrochloride; and
c) optionally, heating Omecamtiv mecarbil dihydrochloride of step b).

In another aspect, the present application provides a process for the preparation of crystalline form OMT-O of Omecamtiv mecarbil dihydrochloride and Oxalic acid, comprising the step of contacting Omecamtiv mecarbil dihydrochloride with Oxalic acid.

In another aspect, the present application provides a process for the preparation of crystalline form OMT-F of Omecamtiv mecarbil dihydrochloride and Fumaric acid, comprising the step of contacting Omecamtiv mecarbil dihydrochloride with Fumaric acid.

In another aspect, the present application provides a process for the preparation of crystalline form OMT-FS of Omecamtiv mecarbil dihydrochloride, comprising the step of contacting Omecamtiv mecarbil dihydrochloride with formamide.

In another aspect, the present application provides a pharmaceutical composition comprising crystalline and/or amorphous solid dispersions of Omecamtiv mecarbil dihydrochloride, wherein the crystalline Omecamtiv mecarbil dihydrochloride is selected from the group consisting of crystalline Form DP1, Form DP2, Form DP3, Form DP4, Form OMT-L, OMT-D, OMT-O, OMT-F, OMT-FS and mixtures thereof, and atleast one pharmaceutically acceptable excipient.

### BRIEF DESCRIPTION OF THE DRAWING

FIG. 1 is an illustrative X-ray powder diffraction pattern of amorphous form of Omecamtiv mecarbil dihydrochloride prepared by the method of Example No 1.

FIG. 2 is an illustrative X-ray powder diffraction pattern of amorphous solid dispersion of Omecamtiv mecarbil dihydrochloride and PVP K-30 prepared by the method of Example No 2.

FIG. 3 is an illustrative X-ray powder diffraction pattern of amorphous solid dispersion of Omecamtiv mecarbil dihydrochloride and co-povidone prepared by the method of Example No 3.

FIG. 4 is an illustrative X-ray powder diffraction pattern of Omecamtiv mecarbil dihydrochloride monohydrate form A prepared by the method of Example No 4.

FIG. 5 is an illustrative X-ray powder diffraction pattern of premix of amorphous Omecamtiv mecarbil dihydrochloride with Syloid prepared by the method of Example No 5.

FIG. 6 is an illustrative X-ray powder diffraction pattern of premix of amorphous solid dispersion of Omecamtiv mecarbil dihydrochloride and PVP K-30 with Syloid prepared by the method of Example No 6.

FIG. 7 is an illustrative X-ray powder diffraction pattern of premix of amorphous solid dispersion of Omecamtiv mecarbil dihydrochloride and co-povidone with Syloid prepared by the method of Example No 7.

FIG. 8 is an illustrative X-ray powder diffraction pattern of premix of amorphous solid dispersion of Omecamtiv mecarbil dihydrochloride and Soluplus with Syloid prepared by the method of Example No 8.

FIG. 9 is an illustrative X-ray powder diffraction pattern of crystalline form DP1 of Omecamtiv mecarbil dihydrochloride prepared by the method of Example No 9.

FIG. 10 is an illustrative X-ray powder diffraction pattern of crystalline form DP2 of Omecamtiv mecarbil dihydrochloride prepared by the method of Example No 10.

FIG. 11 is an illustrative X-ray powder diffraction pattern of crystalline form DP3 of Omecamtiv mecarbil dihydrochloride prepared by the method of Example No 11.

FIG. 12 is an illustrative X-ray powder diffraction pattern of crystalline form DP4 of Omecamtiv mecarbil dihydrochloride prepared by the method of Example No 12.

FIG. 13 is an illustrative X-ray powder diffraction pattern of crystalline form OMT-L of Omecamtiv mecarbil dihydrochloride prepared by the method of Example No 13.

FIG. 14 is an illustrative X-ray powder diffraction pattern of crystalline form OMT-D of Omecamtiv mecarbil dihydrochloride prepared by the method of Example No 14.

FIG. 15 is an illustrative X-ray powder diffraction pattern of crystalline form OMT-O of Omecamtiv mecarbil dihydrochloride and Oxalic acid prepared by the method of Example No 15.

FIG. 16 is an illustrative thermogram of crystalline form OMT-D of Omecamtiv mecarbil dihydrochloride prepared by the method of Example No 14.

FIG. 17 is an illustrative X-ray powder diffraction pattern of crystalline form OMT-F of Omecamtiv mecarbil dihydrochloride and Fumaric acid prepared by the method of Example No 16.

FIG. 18 is an illustrative X-ray powder diffraction pattern of crystalline form OMT-FS of Omecamtiv mecarbil dihydrochloride prepared by the method of Example No 17.

### DETAILED DESCRIPTION

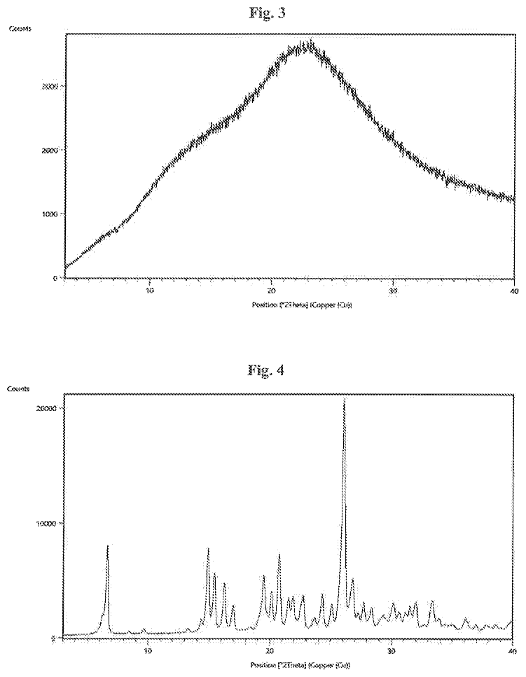

In an aspect, the present application provides an amorphous solid dispersion of Omecamtiv mecarbil or its hydrochloride salt together with at least one pharmaceutically acceptable excipient. In embodiments, the present application provides amorphous solid dispersion of Omecamtiv mecarbil together with at least one pharmaceutically acceptable excipient. In embodiments, present application provides amorphous solid dispersion of Omecamtiv mecarbil dihydrochloride together with at least one pharmaceutically acceptable excipient. In embodiments, the application provides amorphous solid dispersion of Omecamtiv mecarbil dihydrochloride, characterized by a PXRD pattern of FIG. 2 or 3.

In embodiments, at least one pharmaceutically acceptable excipient of this aspect may be selected from the group consisting of polyvinyl pyrrolidone, povidone K-30, povidone K-60, Povidone K-90, polyvinylpyrrolidone vinylacetate, co-povidone NF, polyvinylacetal diethylaminoacetate (AEA®), polyvinyl acetate phthalate, polysorbate 80, polyoxyethylene-polyoxypropylene copolymers (Poloxamer® 188), polyoxyethylene (40) stearate, polyethyene glycol monomethyl ether, polyethyene glycol, poloxamer 188, pluronic F-68, methylcellulose, methacrylic acid copolymer (Eudragit or Eudragit-RLPO), hydroxypropylmethyl cellulose (HPMC), hydroxypropyl cellulose (HPC), hydroxypropylmethyl cellulose phthalate, hydroxypropylmethyl cellulose acetate succinate (HPMC-AS), hydroxypropylmethyl cellulose, hydroxypropyl cellulose SSL (HPC-SSL), hydroxypropyl cellulose SL(HPC-SL), hydroxypropyl cellulose L (HPC-L), hydroxyethyl cellulose, Soluplus® (polyvinyl caprolactam-polyvinyl acetate-polyethylene glycol graft copolymer (PCL-PVAc-PEG)), gelucire 44/14, ethyl cellulose, D-alpha-tocopheryl polyethylene glycol 1000 succinate, cellulose acetate phthalate, carboxymethylethylcelluloseand the like; Solid dispersions of the present application also include the solid dispersions obtained by combining Omecamtiv mecarbil or its hydrochloride with a suitable non-polymeric excipient by employing techniques known in the art or procedures described or exemplified in any aspect of the instant application.

In embodiments, the present application provide an amorphous solid dispersion of Omecamtiv mecarbil dihydrochloride together with polyvinyl pyrrolidone (povidone). In embodiments, the present application provide an amorphous solid dispersion of Omecamtiv mecarbil dihydrochloride together with co-povidone.

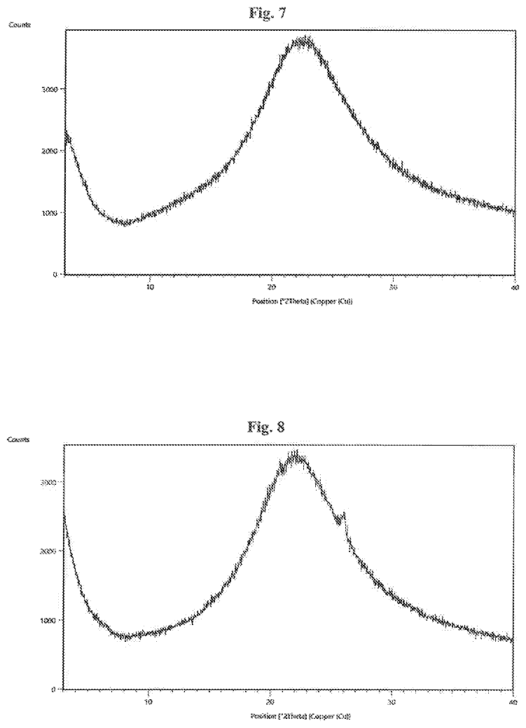

In another aspect, the present application provides a stable amorphous premix comprising an amorphous solid dispersion of Omecamtiv mecarbil or its hydrochloride salt together with at least one pharmaceutically acceptable excipient and Syloid. Syloid may be Syloid 244 FP. In embodiments, amorphous solid dispersion of Omecamtiv mecarbil or its hydrochloride may be combined with Syloid or any other suitable additional pharmaceutically acceptable excipient to obtain premix of this aspect. In embodiments, amorphous solid dispersion of Omecamtiv mecarbil or its hydrochloride may be combined with Syloid or any other suitable additional excipient using a technique known in art or according to the previous aspects of the present application such as grinding together in mortar-pestle. In embodiments, the application provides stable amorphous premix of Omecamtiv mecarbil dihydrochloride with Syloid, characterized by a PXRD pattern of FIG. 6 or 7.

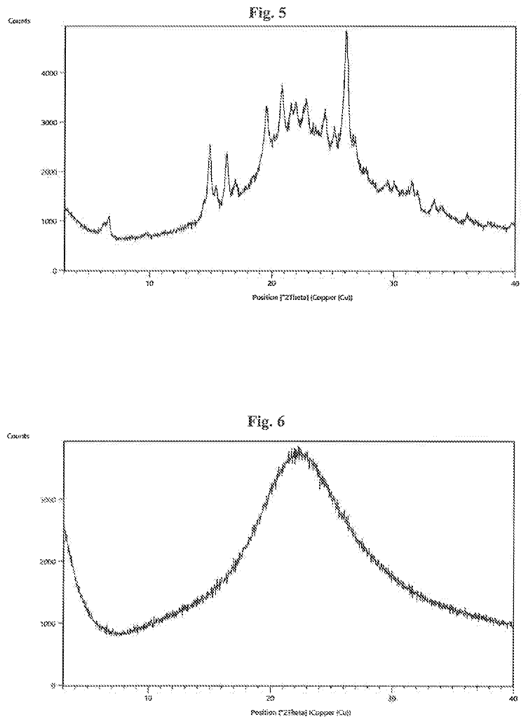

Inventors of present application have found that the amorphous solid dispersion of Omecamtiv mecarbil dihydrochloride together with pharmaceutically acceptable excipient and its premix with Syloid, wherein excipient is selected from the group consisting of polyvinyl pyrrolidone (povidone) and co-povidone, are stable for atleast 2 weeks stored in a closed container at ambient temperature conditions. While the amorphous form of Omecamtiv mecarbil dihydrochloride and its premix with Syloid was found to be unstable as depicted in FIG. 5. Further, the premix of amorphous solid dispersion with Soluplus is also unstable as depicted in FIG. 8.

In another aspect, the present application provides a crystalline Form DP1 of Omecamtiv mecarbil dihydrochloride, characterized by a PXRD pattern comprising the peaks at about 6.55, 16.69, 25.53 and 26.42±0.2° 2θ. In embodiments, the crystalline Form DP1 of Omecamtiv mecarbil dihydrochloride is characterized by one or more additional peaks at about 20.42 and 19.67° 2θ. In embodiments, the application provides crystalline Form DP1 of Omecamtiv mecarbil dihydrochloride, characterized by a PXRD pattern of FIG. 9.

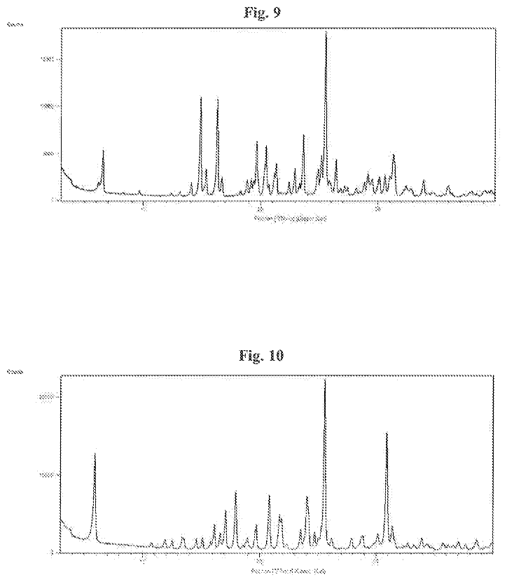

In another aspect, the present application provides a crystalline Form DP2 of Omecamtiv mecarbil dihydrochloride, characterized by a PXRD pattern comprising the peaks at about 5.89, 17.94, 25.58 and 30.88±0.2° 2θ. In embodiments, the crystalline Form DP2 of Omecamtiv mecarbil dihydrochloride is characterized by one or more additional peaks at about 16.13, 17.07, 20.8 and 24.74° 2θ. In embodiments, the application provides crystalline Form DP2 of Omecamtiv mecarbil dihydrochloride, characterized by a PXRD pattern of FIG. 10.

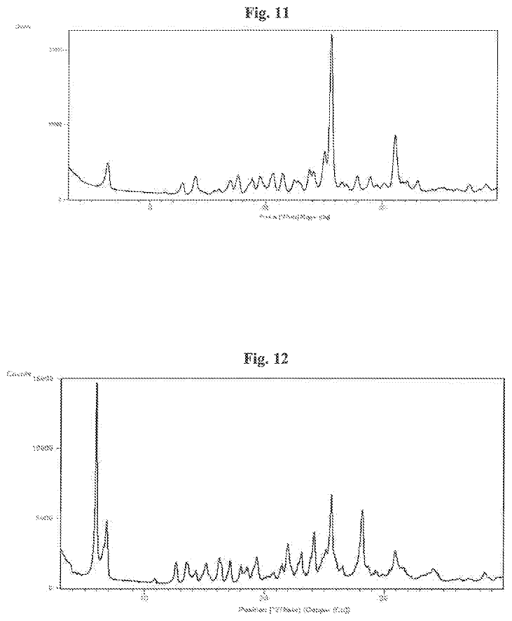

In another aspect, the present application provides a crystalline Form DP3 of Omecamtiv mecarbil dihydrochloride, characterized by a PXRD pattern comprising the peaks at about 25.64, 13.93 and 31.17±0.2° 2θ. In embodiments, the crystalline Form DP3 of Omecamtiv mecarbil dihydrochloride is characterized by one or more additional peaks at about 6.34 and 17.6° 2θ. In embodiments, the application provides crystalline Form DP3 of Omecamtiv mecarbil dihydrochloride, characterized by a PXRD pattern of FIG. 11.

In another aspect, the present application provides a crystalline Form DP4 of Omecamtiv mecarbil dihydrochloride, characterized by a PXRD pattern comprising the peaks at about 6.8, 12.62 and 18.54±0.2° 2θ. In embodiments, the crystalline Form DP4 of Omecamtiv mecarbil dihydrochloride is characterized by one or more additional peaks at about 5.97 18.06, 23.04, 25.61, 28.16 and 30.95° 2θ. In embodiments, the application provides crystalline Form DP4 of Omecamtiv mecarbil dihydrochloride, characterized by a PXRD pattern of FIG. 12.

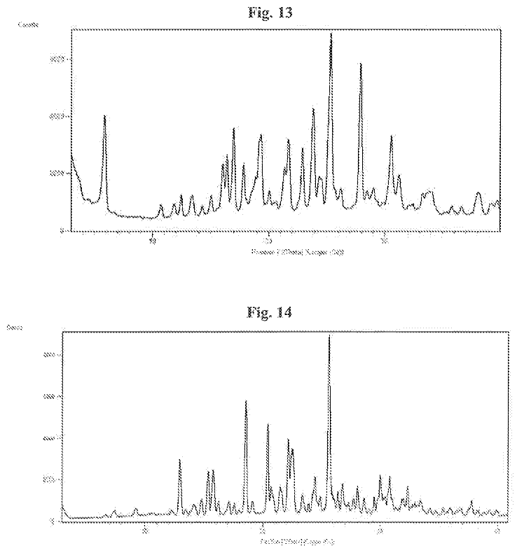

In another aspect, the present application provides a crystalline Form OMT-L of Omecamtiv mecarbil dihydrochloride, characterized by a PXRD pattern comprising the peaks at about 5.87, 17.84, 23.8, 25.35 and 27.91°±0.2° 2θ. In embodiments, the crystalline Form OMT-L of Omecamtiv mecarbil dihydrochloride is characterized by one or more additional peaks at about 30.56 and 22.91° 2θ. In embodiments, the application provides crystalline Form OMT-L of Omecamtiv mecarbil dihydrochloride, characterized by a PXRD pattern of FIG. 13. In embodiments, the crystalline Form OMT-L of Omecamtiv mecarbil dihydrochloride with Lactic acid.

In another aspect, the present application provides a crystalline Form OMT-D of Omecamtiv mecarbil dihydrochloride, characterized by a PXRD pattern comprising the peaks at about 12.98, 15.79, 18.55 and 25.63°±0.2° 2θ. In embodiments, the crystalline Form OMT-D of Omecamtiv mecarbil dihydrochloride is characterized by an additional peak at about 20.41°±0.2° 2θ. In embodiments, the application provides crystalline Form OMT-D of Omecamtiv mecarbil dihydrochloride, characterized by a PXRD pattern of FIG. 14. In embodiments, crystalline Form OMT-D of Omecamtiv mecarbil dihydrochloride is a solvate with dimethyl sulfoxide. In embodiments, crystalline Form OMT-D is monosolvate with dimethyl sulfoxide. In embodiments, crystalline Form OMT-D is stable for atleast 45 days stored in a closed container. In embodiments, the crystalline Form OMT-D is characterized by a thermogram of FIG. 16.

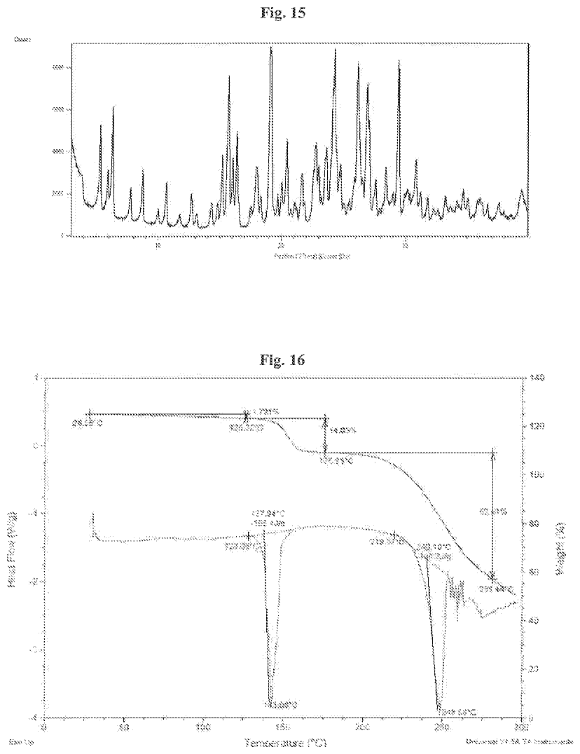

In another aspect, the present application provides a crystalline Form OMT-O of Omecamtiv mecarbil dihydrochloride and oxalic acid, characterized by a PXRD pattern comprising the peaks at about 15.69, 24.26 and 29.44±0.2° 2θ. In embodiments, the crystalline Form OMT-O of Omecamtiv mecarbil dihydrochloride and Oxalic acid is characterized by one or more additional peaks at about 5.32, 12.71, 6.32 and 16.39°±0.2° 2θ. In embodiments, the application provides crystalline Form OMT-O of Omecamtiv mecarbil dihydrochloride and Oxalic acid, characterized by a PXRD pattern of FIG. 15.

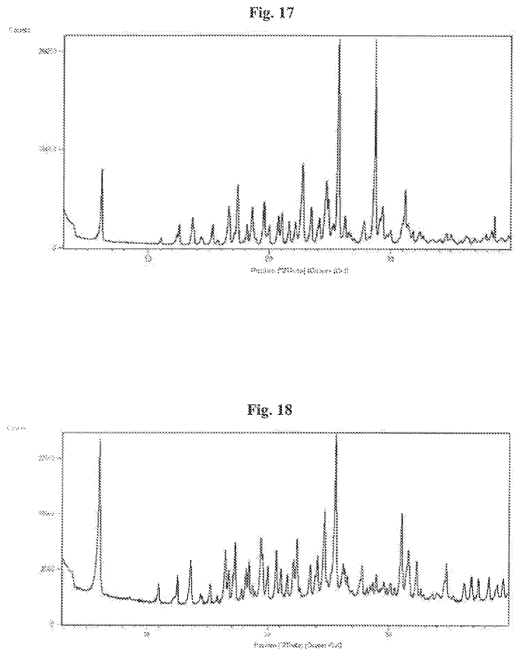

In another aspect, the present application provides a crystalline Form OMT-F of Omecamtiv mecarbil dihydrochloride and Fumaric acid, characterized by a PXRD pattern comprising the peaks at about 6.14 and 17.37°±0.2° 2θ. In embodiments, the crystalline Form OMT-F of Omecamtiv mecarbil dihydrochloride and Fumaric acid is characterized by one or more additional peaks at about 16.65, 23.45 and 25.71°±0.2° 2θ. In embodiments, the application provides crystalline Form OMT-F of Omecamtiv mecarbil dihydrochloride and Fumaric acid, characterized by a PXRD pattern of FIG. 17.

In another aspect, the present application provides a crystalline Form OMT-FS of Omecamtiv mecarbil dihydrochloride characterized by a PXRD pattern comprising the peaks at about 24.68, 25.61 and 31.06°±0.2° 2θ. In embodiments, the crystalline Form OMT-FS of Omecamtiv mecarbil dihydrochloride is characterized by an additional peaks at about 6.07°±0.2° 2θ. In embodiments, the application provides crystalline Form OMT-FS of Omecamtiv mecarbil dihydrochloride, characterized by a PXRD pattern of FIG. 18.

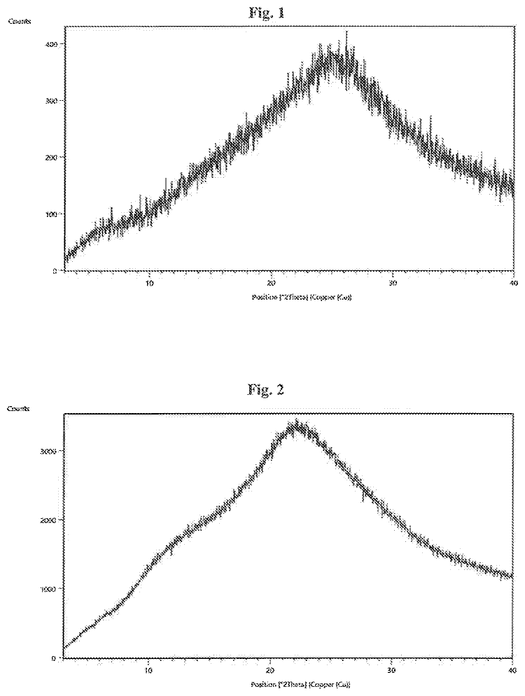

In another aspect, the present application provides amorphous Omecamtiv mecarbil or its hydrochloride salt. In embodiments, the present application provides amorphous form of Omecamtiv mecarbil. In embodiments, the present application provides amorphous form of Omecamtiv mecarbil dihydrochloride. Amorphous form of Omecamtiv mecarbil dihydrochloride is characterized by the X-ray diffraction pattern as depicted in FIG. 1.

In another aspect, the present application provides a process for the preparation of amorphous Omecamtiv mecarbil and its hydrochloride salt, comprising the step of providing a solution of Omecamtiv mecarbil or its hydrochloride salt in a solvent and removing the solvent. In embodiments, the present application provides a process for the preparation of amorphous Omecamtiv mecarbil. In embodiments, the present application provides a process for the preparation of amorphous Omecamtiv mecarbil dihydrochloride.

In embodiments, solvent may be selected from the group consisting of methanol, ethanol, 2-propanol, 1-butanol, 2-butanol, 1-pentanol, 2-pentanol, 3-pentanol, dichloromethane; tetrahydrofuran, 1,4-dioxane, acetone, methyl ethyl ketone, methyl isobutyl ketone; methyl acetate, ethyl acetate, isopropyl acetate, water and mixtures thereof.

In embodiments, providing a solution of Omecamtiv mecarbil or its hydrochloride salt may be carried out by dissolving Omecamtiv mecarbil or its dihydrochloride in a solvent or by directly taking the reaction mixture containing Omecamtiv mecarbil or its dihydrochloride in a solvent, optionally under heating. In embodiments, a solution of Omecamtiv mecarbil or its dihydrochloride may be filtered to make it clear and free of unwanted particles. In embodiments, the obtained solution may be optionally treated with an adsorbent material, such as carbon and/or hydrose, to remove colored components, etc., before filtration.

In an embodiment, removal of solvent may be carried out by methods known in the art or any procedure disclosed in the present application. In preferred embodiments, removal of solvent may include, but not limited to: solvent evaporation under atmospheric pressure or reduced pressure/vacuum such as a rotational distillation using Büchi® Rotavapor®, spray drying, freeze drying, thin film drying, agitated thin film drying, rotary vacuum paddle dryer (RVPD) and the like.

In preferred embodiment, the solvent may be removed under reduced pressures and at temperatures of less than about 100° C., less than about 80° C., less than about 40° C., less than about 20° C., less than about 0° C., less than about −20° C., less than about −40° C., less than about −60° C., less than about −80° C., or any other suitable temperatures.

In embodiments, the isolation of an amorphous form of Omecamtiv mecarbil or its dihydrochloride involves recovering the solid obtained. The solid may be recovered using techniques such as by scraping, or by shaking the container, or triturating with a solvent to make slurry followed by filtration, or other techniques specific to the equipment used. In an embodiment, the amorphous form of Omecamtiv mecarbil or its dihydrochloride obtained may be optionally dried before or after isolating it.

Amorphous form of Omecamtiv mecarbil or dihydrochloride isolated may be dried in suitable drying equipment such as vacuum oven, rotatory cone dryer, air oven, fluidized bed dryer, spin flash dryer, flash dryer, or the like. The drying may be carried out at atmospheric pressure or under reduced pressures at temperatures of less than about 100° C., less than about 60° C., less than about 40° C., or any other suitable temperatures. The drying may be carried out for any time period required for obtaining a desired quality, such as from about 15 minutes to 10 hours or longer.

Amorphous form of Omecamtiv mecarbil or its dihydrochloride obtained may be optionally combined with at least one pharmaceutically acceptable excipient. In an embodiment, amorphous form of Omecamtiv mecarbil or its dihydrochloride may be combined with excipient to form a premix using a technique known in art or by the procedures disclosed in the present application. Pharmaceutically acceptable excipient may be selected from the group consisting of silicon dioxide, e.g. colloidal or fumed silicon dioxide or porous silica or Syloid; copolymers, such as polyethylene/polyvinyl alcohol copolymer, polyethylene/polyvinyl pyrrolidinone copolymer; and cellulose, preferably microcrystalline cellulose.

In embodiments, amorphous form of Omecamtiv mecarbil or its dihydrochloride may be combined with excipient either by physical blending of both the solid components or by suspending both the components in a suitable solvent and conditions, such that both the components remain unaffected. Blending may be carried out using techniques known in art such as grinding in mortar-pestle, mixing in rotatory cone dryer, fluidized bed dryer or the like optionally under reduced pressure/vacuum or inert atmosphere such nitrogen at suitable temperature and sufficient time to obtain uniform premix of amorphous form of Omecamtiv mecarbil or its dihydrochloride and at least one pharmaceutically acceptable excipient.

In another aspect, the present application provides a process for the preparation of amorphous solid dispersion of Omecamtiv mecarbil or its hydrochloride salt, comprising the step of providing a solution of Omecamtiv mecarbil or its hydrochloride salt together with at least one pharmaceutically acceptable excipient in a solvent and removing the solvent. In embodiments, the process produces amorphous solid dispersion of Omecamtiv mecarbil. In embodiments, the process produces amorphous solid dispersion of Omecamtiv mecarbil dihydrochloride.

In embodiments, the solvent and the excipient of this aspect may be selected from the group of solvents and excipients of the previous aspect.

In embodiments, providing a solution may be carried out by dissolving Omecamtiv mecarbil or its hydrochloride and at least one pharmaceutically acceptable excipient simultaneously or separately in same or different solvents. In embodiments, providing a solution may be carried as described in previous aspect. In embodiments, a solution of Omecamtiv mecarbil or its hydrochloride and the excipient may be prepared at any suitable temperatures, such as about 0° C. to about the reflux temperature of the solvent used. Stirring and heating may be used to reduce the time required for the dissolution process.

In embodiments, removal of solvent may be carried out by methods known in the art or any procedure disclosed in previous aspect of the present application. In preferred embodiments, removal of solvent may include, but not limited to: solvent evaporation under atmospheric pressure or reduced pressure/vacuum such as a rotational distillation using Büchi® Rotavapor®, spray drying, freeze drying, agitated thin film drying and the like.

In embodiments, the isolation and drying of the amorphous solid dispersion may be carried out by known in the art or any procedure disclosed in previous aspect of the present application.

Amorphous form of Omecamtiv mecarbil or its hydrochloride or its solid dispersion of present application may be obtained alternatively either by employing a melt-extrusion technique or by combining a solution of Omecamtiv mecarbil or its hydrochloride as obtained any of the aspects of present application with a suitable anti-solvent. In embodiments, amorphous product may be obtained by employing suitable melt-extrusion conditions or any of the procedures known in the art for obtaining amorphous product by melt-extrusion technique. In embodiments, solution of Omecamtiv mecarbil or its hydrochloride may be combined with the anti-solvent at suitable temperature and for sufficient time to obtain amorphous product. Suitable anti-solvent is a solvent, wherein Omecamtiv mecarbil or its hydrochloride has low solubility.

In another aspect, the present application provides a process for the preparation of crystalline Omecamtiv mecarbil dihydrochloride, comprising a step of contacting the solution of Omecamtiv mecarbil dihydrochloride in solvent with an anti-solvent.

In embodiments, the crystalline Omecamtiv mecarbil dihydrochloride is anhydrous. In embodiments, the crystalline Omecamtiv mecarbil dihydrochloride is a solvate. In embodiments, the solvate of Omecamtiv mecarbil dihydrochloride is a hydrate. In embodiments, the crystalline Omecamtiv mecarbil dihydrochloride is selected from the group comprising of monohydrate Form A, anhydrous form B and anhydrous form C. In embodiments, the crystalline Omecamtiv mecarbil dihydrochloride is monohydrate Form A.

In embodiments, solution of Omecamtiv mecarbil dihydrochloride may be obtained either by dissolving Omecamtiv mecarbil dihydrochloride in a solvent or in situ by combining Omecamtiv mecarbil with hydrochloric acid in the presence of a solvent. In embodiments, solvent may include but not limited to water, dimethyl sulfoxide, dimethylformamide, N-methylpyrrolidone, methanol, ethanol, 2-propanol, dichloromethane and mixtures thereof.

In embodiments, the anti-solvent is a solvent in which Omecamtiv mecarbil has minimum solubility or insoluble. Anti-solvent may include but not limited to acetonitrile, methyl acetate, ethyl acetate, isopropyl acetate, acetone, methyl ethyl ketone, methyl isobutyl ketone, tetrahydrofuran, 1-4-dioxane, hexane and mixtures thereof.

In embodiments, the solution of Omecamtiv mecarbil dihydrochloride may be contacted at suitable temperature of about 0° C. or above. In embodiments, the solution of Omecamtiv mecarbil dihydrochloride may be contacted with anti-solvent rapidly in single lot or gradually in multiple lots.

In embodiments, the crystalline Omecamtiv mecarbil dihydrochloride may be recovered using techniques such as filtration, decantation of solvents or any other suitable techniques. In embodiments, the crystalline Omecamtiv mecarbil dihydrochloride may be optionally dried under suitable drying conditions.

In another aspect, the present application provides a process for the preparation of crystalline form DP1 of Omecamtiv mecarbil dihydrochloride, comprising the step of contacting Omecamtiv mecarbil dihydrochloride with formic acid.

In embodiments, the Omecamtiv mecarbil dihydrochloride may be either in crystalline or in amorphous states. In embodiments, the crystalline Omecamtiv mecarbil may be in any crystalline form reported in the literature such as hydrated form A, anhydrous forms B or C.

In embodiments, Omecamtiv mecarbil dihydrochloride may be contacted in the form of a solution or suspension containing Omecamtiv mecarbil dihydrochloride in formic acid or in mixture of formic acid and any other miscible solvent.

In embodiments, Omecamtiv mecarbil dihydrochloride may be contacted with formic acid at a suitable temperature of about 0° C. to reflux temperature of the mixture.

In embodiments, contacting may be carried out by dissolving Omecamtiv mecarbil dihydrochloride in formic acid or a mixture thereof, optionally under heating. In alternate embodiments, contacting may be carried out by directly combining a reaction mixture containing Omecamtiv mecarbil and hydrochloric acid with formic acid or a mixture thereof.

In embodiments, the solution containing Omecamtiv mecarbil dihydrochloride and formic acid may be cooled to a suitable temperature of about 30° C. or below. In embodiments, the solution may be optionally contacted with a suitable anti-solvent. Anti-solvent is a solvent in which Omecamtiv mecarbil dihydrochloride is insoluble or low soluble. In embodiments, the solution containing Omecamtiv mecarbil dihydrochloride may be evaporated at a suitable temperature, optionally under reduced pressure.

In embodiments, the crystalline form DP1 of Omecamtiv mecarbil dihydrochloride obtained may be separated according to any suitable techniques know in the art such as filtration or centrifugation. In embodiments, the crystalline form DP1 may be dried under suitable drying conditions such as aerial drying, vacuum drying or rotatory vacuum drying. The crystalline form DP1 of Omecamtive mecarbil dihydrochloride obtained according to the process of this aspect may be characterized by a PXRD pattern comprising the peaks at about 6.55, 16.69, 25.53 and 26.42±0.2° 2θ.

In another aspect, the present application provides a process for the preparation of crystalline form DP2 of Omecamtiv mecarbil dihydrochloride, comprising the step of contacting Omecamtiv mecarbil dihydrochloride with acetic acid.

In embodiments, the Omecamtiv mecarbil dihydrochloride may be either in crystalline or in amorphous states. In embodiments, the crystalline Omecamtiv mecarbil may be in any crystalline form reported in the literature such as hydrated form A, anhydrous forms B or C.

In embodiments, Omecamtiv mecarbil dihydrochloride may be contacted in the form of a solution or suspension containing Omecamtiv mecarbil dihydrochloride in acetic acid or in mixture of acetic acid and any other miscible solvent.

In embodiments, Omecamtiv mecarbil dihydrochloride may be contacted with acetic acid at a suitable temperature of about 0° C. to reflux temperature of the mixture.

In embodiments, contacting may be carried out by dissolving Omecamtiv mecarbil dihydrochloride in acetic acid or a mixture thereof, optionally under heating. In alternate embodiments, contacting may be carried out by directly combining a reaction mixture containing Omecamtiv mecarbil and hydrochloric acid with acetic acid or a mixture thereof.

In embodiments, the solution containing Omecamtiv mecarbil dihydrochloride and acetic acid may be cooled to a suitable temperature of about 30° C. or below. In embodiments, the solution of Omecamtiv mecarbil dihydrochloride and acetic acid may be optionally contacted with a suitable anti-solvent. Anti-solvent is a solvent in which Omecamtiv mecarbil dihydrochloride is insoluble or low soluble. In embodiments, the solution containing Omecamtiv mecarbil dihydrochloride may be evaporated at a suitable temperature, optionally under reduced pressure.

In embodiments, the crystalline form DP2 of Omecamtiv mecarbil dihydrochloride obtained by the process of this aspect may be separated according to any suitable techniques know in the art such as filtration or centrifugation. In embodiments, the crystalline form DP2 may be dried under suitable drying conditions such as aerial drying, vacuum drying or rotatory vacuum drying. The crystalline form DP2 of Omecamtive mecarbil dihydrochloride obtained according to the process of this aspect may be characterized by a PXRD pattern comprising the peaks at about 5.89, 17.94, 25.58 and 30.88±0.2° 2θ.

In another aspect, the present application provides a process for the preparation of crystalline form DP3 of Omecamtiv mecarbil dihydrochloride, comprising the step of heating crystalline form DP2 of Omecamtiv mecarbil dihydrochloride.

In embodiments, heating crystalline form DP2 of Omecamtiv mecarbil dihydrochloride may be carried out under suitable conditions such as controlled rate of heating and under vacuum or inert gas atmosphere such as nitrogen.

In embodiments, heating crystalline form DP2 may be carried out in a suitable drying equipment, such as air or vacuum tray dryers and in suitable thermal instruments such as thermo gravimetric analyzers or calorimeters.

In embodiments, heating may be carried out at suitable temperature of about 40° C. or above and for sufficient time to complete the conversion of crystalline form DP2 to DP3. In embodiments, the crystalline form DP3 of Omecamtiv mecarbil obtained by the process of this aspect may be characterized by a PXRD pattern comprising the peaks at about 25.64, 13.93 and 31.17±0.2° 2θ.

In another aspect, the present application provides a process for the preparation of crystalline form DP4 of Omecamtiv mecarbil dihydrochloride, comprising the steps of:
a) contacting Omecamtiv mecarbil dihydrochloride with propionic acid;
b) isolating Omecamtiv mecarbil dihydrochloride; and
c) optionally, heating Omecamtiv mecarbil dihydrochloride of step b).

In embodiments, the Omecamtiv mecarbil dihydrochloride may be either in crystalline or in amorphous states. In embodiments, the crystalline Omecamtiv mecarbil may be in any crystalline form reported in the literature.

In embodiments, Omecamtiv mecarbil dihydrochloride may be contacted in the form of a solution or suspension containing Omecamtiv mecarbil dihydrochloride in propionic acid or in mixture of propionic acid and any other miscible solvent.

In embodiments, Omecamtiv mecarbil dihydrochloride may be contacted with propionic acid at a suitable temperature of about 0° C. to reflux temperature of the mixture.

In embodiments, contacting may be carried out by dissolving Omecamtiv mecarbil dihydrochloride in propionic acid or a mixture thereof, optionally under heating. In alternate embodiments, contacting may be carried out by directly combining a reaction mixture containing Omecamtiv mecarbil and hydrochloric acid with propionic acid or a mixture thereof.

In embodiments, the solution containing Omecamtiv mecarbil dihydrochloride and propionic acid may be cooled to a suitable temperature of about 30° C. or below. In embodiments, the solution may be optionally contacted with a suitable anti-solvent. Anti-solvent is a solvent in which Omecamtiv mecarbil dihydrochloride is insoluble or low soluble. In embodiments, the solution containing Omecamtiv mecarbil dihydrochloride may be evaporated at a suitable temperature, optionally under reduced pressure.

In embodiments, step b) of this aspect may be carried out by isolating the Omecamtiv mecarbil dihydrochloride. The isolation of Omecamtiv mecarbil dihydrochloride may be carried out according to any suitable techniques know in the art such as filtration or centrifugation.

In embodiments, step c) of this aspect may be carried out by heating Omecamtiv mecarbil dihydrochloride of step b) under suitable conditions such as controlled rate of heating and under vacuum or inert gas atmosphere such as nitrogen. In embodiments, heating may be carried out in a suitable drying equipment, such as air or vacuum tray dryers and in suitable thermal instruments such as thermo gravimetric analyzers or calorimeters.

In embodiments, heating may be carried out at suitable temperature of about 60° C. and above and for sufficient time to complete the conversion of Omecamtiv mecarbil dihydrochloride of step b) to crystalline form DP4. The crystalline form DP4 of Omecamtive mecarbil dihydrochloride obtained according to the process of this aspect may be characterized by a PXRD pattern comprising the peaks at about 6.8, 12.62 and 18.54±0.2° 2θ.

In another aspect, the present application provides a process for the preparation of crystalline form OMT-L of Omecamtiv mecarbil dihydrochloride, comprising the steps of
a) contacting Omecamtiv mecarbil dihydrochloride with DL-Lactic acid
b) isolating Omecamtiv mecarbil dihydrochloride; and
c) optionally, heating Omecamtiv mecarbil dihydrochloride of step b).

In embodiments, the Omecamtiv mecarbil dihydrochloride may be either in crystalline or in amorphous states. In embodiments, the crystalline Omecamtiv mecarbil may be in any crystalline form reported in the literature.

In embodiments, Omecamtiv mecarbil dihydrochloride may be contacted in the form of a solution or suspension containing Omecamtiv mecarbil dihydrochloride in DL-lactic acid or in mixture of DL-lactic acid and any other miscible solvent.

In embodiments, Omecamtiv mecarbil dihydrochloride may be contacted with DL-lactic acid at a suitable temperature of about 0° C. to reflux temperature of the mixture.

In embodiments, contacting may be carried out by dissolving Omecamtiv mecarbil dihydrochloride in DL-lactic acid or a mixture thereof, optionally under heating. In alternate embodiments, contacting may be carried out by directly combining a reaction mixture containing Omecamtiv mecarbil and hydrochloric acid with DL-lactic acid or a mixture thereof.

In embodiments, the solution containing Omecamtiv mecarbil dihydrochloride and DL-lactic acid may be cooled to a suitable temperature of about 30° C. or below. In embodiments, the solution may be optionally contacted with a suitable anti-solvent. Anti-solvent is a solvent in which Omecamtiv mecarbil dihydrochloride is insoluble or low soluble. In an embodiment, suitable anti-solvent may include, but not limited to diethyl ether, methyl tert. butyl ether, tetrahydrofuran and 1,4-dioxane. In embodiments, the solution containing Omecamtiv mecarbil dihydrochloride may be evaporated at a suitable temperature, optionally under reduced pressure.

In embodiments, step b) of this aspect may be carried out by isolating the Omecamtiv mecarbil dihydrochloride. The isolation of Omecamtiv mecarbil dihydrochloride may be carried out according to any suitable techniques know in the art such as filtration or centrifugation.

In embodiments, step c) of this aspect may be carried out by heating Omecamtiv mecarbil dihydrochloride of step b) under suitable conditions such as controlled rate of heating and under vacuum or inert gas atmosphere such as nitrogen. In embodiments, heating may be carried out in a suitable drying equipment, such as air or vacuum tray dryers and in suitable thermal instruments such as thermo gravimetric analyzers or calorimeters.

In embodiments, heating may be carried out at suitable temperature of about 30° C. and above and for sufficient time to complete the conversion of Omecamtiv mecarbil dihydrochloride of step b) to crystalline form OMT-L. The crystalline form OMT-L of Omecamtive mecarbil dihydrochloride obtained according to the process of this aspect may be characterized by a PXRD pattern comprising the peaks at about 5.87, 17.84, 23.8, 25.35 and 27.91°±0.2° 2θ.

In another aspect, the present application provides a process for the preparation of crystalline form OMT-D of Omecamtiv mecarbil dihydrochloride, comprising the steps of
a) contacting Omecamtiv mecarbil dihydrochloride with dimethyl sulfoxide;
b) isolating Omecamtiv mecarbil dihydrochloride; and
c) optionally, heating Omecamtiv mecarbil dihydrochloride of step b).

In embodiments, the Omecamtiv mecarbil dihydrochloride used in this aspect may be either in crystalline or in amorphous states. In embodiments, the crystalline Omecamtiv mecarbil may be in any of the crystalline forms reported in the literature.

In embodiments, Omecamtiv mecarbil dihydrochloride may be contacted in the form of a solution or suspension containing Omecamtiv mecarbil dihydrochloride in dimethyl sulfoxide or in mixture of dimethyl sulfoxide and any other miscible solvent.

In embodiments, Omecamtiv mecarbil dihydrochloride may be contacted with dimethyl sulfoxide at a suitable temperature of about 0° C. to reflux temperature of the mixture.

In embodiments, contacting may be carried out by dissolving Omecamtiv mecarbil dihydrochloride in dimethyl sulfoxide or a mixture thereof, optionally under heating. In alternate embodiments, contacting may be carried out by directly combining a reaction mixture containing Omecamtiv mecarbil and hydrochloric acid with dimethyl sulfoxide or a mixture thereof.

In embodiments, the solution containing Omecamtiv mecarbil dihydrochloride and dimethyl sulfoxide may be cooled to a suitable temperature of about 30° C. or below. In embodiments, the solution may be optionally contacted with a suitable anti-solvent. Anti-solvent is a solvent in which Omecamtiv mecarbil dihydrochloride is insoluble or low soluble. In an embodiment, suitable anti-solvent may include, but not limited to diethyl ether, methyl tert. butyl ether, tetrahydrofuran and 1,4-dioxane. In embodiments, the solution containing Omecamtiv mecarbil dihydrochloride may be evaporated at a suitable temperature, optionally under reduced pressure.

In embodiments, step b) of this aspect may be carried out by isolating the Omecamtiv mecarbil dihydrochloride. The isolation of Omecamtiv mecarbil dihydrochloride may be carried out according to any suitable techniques know in the art such as filtration or centrifugation.

In embodiments, step c) of this aspect may be carried out by heating Omecamtiv mecarbil dihydrochloride of step b) under suitable conditions such as controlled rate of heating and under vacuum or inert gas atmosphere such as nitrogen. In embodiments, heating may be carried out in a suitable drying equipment, such as air or vacuum tray dryers and in suitable thermal instruments such as thermo gravimetric analyzers or calorimeters.

In embodiments, heating may be carried out at suitable temperature of about 30° C. and above and for sufficient time to complete the conversion of Omecamtiv mecarbil dihydrochloride of step b) to crystalline form OMT-D. The crystalline form OMT-D of Omecamtive mecarbil dihydrochloride obtained according to the process of this aspect may be characterized by a PXRD pattern comprising the peaks at about 12.98, 15.79, 18.55 and 25.63°±0.2° 2θ.

In another aspect, the present application provides a process for the preparation of crystalline form OMT-O of Omecamtiv mecarbil dihydrochloride and Oxalic acid, comprising the step of contacting Omecamtiv mecarbil dihydrochloride with Oxalic acid.

In embodiments, the Omecamtiv mecarbil dihydrochloride may be either in crystalline or in amorphous states. In embodiments, the crystalline Omecamtiv mecarbil may be in any crystalline form reported in the literature.

In embodiments, contacting Omecamtiv mecarbil dihydrochloride with Oxalic acid may be carried out, optionally in the presence of a suitable solvent. Suitable solvent may include, but not limited to water, chloroform and mixtures thereof.

In embodiments, contacting Omecamtiv mecarbil dihydrochloride with Oxalic acid may be carried out through the formation of solution or suspension containing Omecamtiv mecarbil dihydrochloride and Oxalic acid in a suitable solvent.

In embodiments, contacting Omecamtiv mecarbil dihydrochloride with Oxalic acid may be carried out at a suitable temperature of about 0° C. or above. In embodiments, contacting Omecamtiv mecarbil dihydrochloride with Oxalic acid may be carried out for sufficient time to complete the formation of crystalline form OMT-O. In an embodiment, the contacting Omecamtiv mecarbil dihydrochloride with Oxalic acid may be carried out for at least one hour or longer.

In another embodiment, contacting Omecamtiv mecarbil dihydrochloride with Oxalic acid may be carried out by suspending Omecamtiv mecarbil dihydrochloride and Oxalic acid in a solvent at a suitable temperature.

In another embodiment, crystalline Form OMT-O may be crystallized out from the solution containing Omecamtiv mecarbil dihydrochloride and Oxalic acid in a solvent by cooling the solution to a suitable temperature or by evaporating the solvent optionally under reduced pressure or by treating with a suitable anti-solvent. Anti-solvent is a solvent in which Omecamtiv mecarbil dihydrochloride and/or Oxalic acid are insoluble or less soluble.

In another embodiment, contacting Omecamtiv mecarbil dihydrochloride with Oxalic acid may be carried out by grinding the mixture of Omecamtiv mecarbil dihydrochloride and Oxalic acid, optionally in the presence of a suitable solvent. In embodiments, grinding the mixture may be carried out in a suitable grinding apparatus such as mortar—pestle or a milling apparatus such as ball mill.

In another embodiment, contacting Omecamtiv mecarbil dihydrochloride with Oxalic acid may be carried out by melting the mixture of Omecamtiv mecarbil dihydrochloride and Oxalic acid at a suitable temperature upto the melting points of the components. In embodiments, the melting of the mixture may be carried out either under atmospheric pressure or under reduced pressure, optionally under inert gas atmosphere such as Nitrogen.

In embodiments, crystalline form OMT-O may be isolated from the solvent according to any suitable techniques know in the art such as filtration or centrifugation.

In embodiments, drying crystalline form OMT-O may be carried out under suitable conditions such as aerial drying or drying under vacuum or inert gas atmosphere such as nitrogen. In embodiments, drying may be carried out in a suitable drying equipment, such as air or vacuum tray dryers. The crystalline form OMT-O of Omecamtive mecarbil dihydrochloride and oxalic acid obtained according to the process of this aspect may be characterized by a PXRD pattern comprising the peaks at about 15.69, 24.26 and 29.44±0.2° 2θ.

In another aspect, the present application provides a process for the preparation of crystalline form OMT-F of Omecamtiv mecarbil dihydrochloride and Fumaric acid, comprising the step of contacting Omecamtiv mecarbil dihydrochloride with Fumaric acid.

In embodiments, the Omecamtiv mecarbil dihydrochloride may be either in crystalline or in amorphous states. In embodiments, the crystalline Omecamtiv mecarbil may be in any crystalline form reported in the literature.

In embodiments, contacting Omecamtiv mecarbil dihydrochloride with Fumaric acid may be carried out, optionally in the presence of a suitable solvent. Suitable solvent may include, but not limited to water, chloroform dichloromethane, methanol, acetonitrile and mixtures thereof.

In embodiments, contacting Omecamtiv mecarbil dihydrochloride with Fumaric acid may be carried out through the formation of solution or suspension containing Omecamtiv mecarbil dihydrochloride and Fumaric acid in a suitable solvent.

In embodiments, contacting Omecamtiv mecarbil dihydrochloride with Fumaric acid may be carried out at a suitable temperature of about 0° C. or above. In embodiments, contacting Omecamtiv mecarbil dihydrochloride with Fumaric acid may be carried out for sufficient time to complete the formation of crystalline form OMT-F. In an embodiment, the contacting Omecamtiv mecarbil dihydrochloride with Fumaric acid may be carried out for at least one hour or longer.

In another embodiment, contacting Omecamtiv mecarbil dihydrochloride with Fumaric acid may be carried out by suspending Omecamtiv mecarbil dihydrochloride and Fumaric acid in a solvent at a suitable temperature.

In another embodiment, crystalline Form OMT-F may be crystallized out from the solution containing Omecamtiv mecarbil dihydrochloride and Fumaric acid in a solvent by cooling the solution to a suitable temperature or by evaporating the solvent optionally under reduced pressure or by treating with a suitable anti-solvent. Anti-solvent is a solvent in which Omecamtiv mecarbil dihydrochloride and/or Fumaric acid are insoluble or less soluble.

In another embodiment, contacting Omecamtiv mecarbil dihydrochloride with Fumaric acid may be carried out by grinding the mixture of Omecamtiv mecarbil dihydrochloride and Fumaric acid, optionally in the presence of a suitable solvent. In embodiments, grinding the mixture may be carried out in a suitable grinding apparatus such as mortar—pestle or a milling apparatus such as ball mill.

In another embodiment, contacting Omecamtiv mecarbil dihydrochloride with Fumaric acid may be carried out by melting the mixture of Omecamtiv mecarbil dihydrochloride and Fumaric acid at a suitable temperature upto the melting points of the components. In embodiments, the melting of the mixture may be carried out either under atmospheric pressure or under reduced pressure, optionally under inert gas atmosphere such as Nitrogen.

In embodiments, crystalline form OMT-F may be isolated from the solvent according to any suitable techniques know in the art such as filtration or centrifugation.

In embodiments, drying crystalline form OMT-F may be carried out under suitable conditions such as aerial drying or drying under vacuum or inert gas atmosphere such as nitrogen. In embodiments, drying may be carried out in a suitable drying equipment, such as air or vacuum tray dryers. The crystalline form OMT-F of Omecamtive mecarbil dihydrochloride and Fumaric acid obtained according to the process of this aspect may be characterized by a PXRD pattern comprising the peaks at about 6.14 and 17.37°±0.2° 2θ.

In another aspect, the present application provides a process for the preparation of crystalline form OMT-FS of Omecamtiv mecarbil dihydrochloride, comprising the step of contacting Omecamtiv mecarbil dihydrochloride with formamide.

In embodiments, the Omecamtiv mecarbil dihydrochloride may be either in crystalline or in amorphous states. In embodiments, the crystalline Omecamtiv mecarbil may be in any crystalline form reported in the literature.

In embodiments, contacting Omecamtiv mecarbil dihydrochloride with formamide may be carried out through the formation of solution or suspension containing Omecamtiv mecarbil dihydrochloride in formamide.

In embodiments, contacting Omecamtiv mecarbil dihydrochloride with formamide may be carried out at a suitable temperature of about 0° C. or above. In embodiments, contacting Omecamtiv mecarbil dihydrochloride with formamide may be carried out for sufficient time to complete the formation of crystalline form OMT-FS. In an embodiment, the contacting Omecamtiv mecarbil dihydrochloride with formamide may be carried out for at least one hour or longer.

In another embodiment, contacting Omecamtiv mecarbil dihydrochloride with formamide may be carried out by suspending Omecamtiv mecarbil dihydrochloride in formamide at a suitable temperature.

In another embodiment, crystalline Form OMT-FS may be crystallized out from the solution containing Omecamtiv mecarbil dihydrochloride in formamide by cooling the solution to a suitable temperature or by evaporating the solvent optionally under reduced pressure or by treating with a suitable anti-solvent. Anti-solvent is a solvent in which Omecamtiv mecarbil dihydrochloride are insoluble or less soluble. Suitable anti-solvent may include, but not limited to diethyl ether, di isopropyl ether, tetrahydrofuran, 1,4-dioxane, water and mixture thereof.

In another embodiment, contacting Omecamtiv mecarbil dihydrochloride with formamide may be carried out by grinding Omecamtiv mecarbil dihydrochloride in the presence of formamide. In embodiments, grinding the mixture may be carried out in a suitable grinding apparatus such as mortar—pestle or a milling apparatus such as ball mill.

In embodiments, crystalline form OMT-FS may be isolated according to any suitable techniques know in the art such as filtration or centrifugation.

In embodiments, drying crystalline form OMT-FS may be carried out under suitable conditions such as aerial drying or drying under vacuum or inert gas atmosphere such as nitrogen. In embodiments, drying may be carried out in a suitable drying equipment, such as air or vacuum tray dryers. The crystalline form OMT-FS of Omecamtiv mecarbil dihydrochloride obtained according to the process of this aspect may be characterized by a PXRD pattern comprising the peaks at about 24.68, 25.61 and 31.06°±0.2° 2θ.

In another aspect, the present application provides a pharmaceutical composition comprising amorphous and/or crystalline Omecamtiv mecarbil dihydrochloride selected from the group consisting of crystalline forms DP1, DP2, DP3, DP4, OMT-L, OMT-D, OMT-O, OMT-F, OMT-FS and mixtures thereof and atleast one pharmaceutically acceptable excipient.

Certain specific aspects and embodiments of the present application will be explained in greater detail with reference to the following examples, which are provided only for purposes of illustration and should not be construed as limiting the scope of the application in any manner. Variations of the described procedures, as will be apparent to those skilled in the art, are intended to be within the scope of the present application.

### Definitions

The term “about” when used in the present application preceding a number and referring to it, is meant to designate any value which lies within the range of ±10%, preferably within a range of ±5%, more preferably within a range of ±2%, still more preferably within a range of ±1% of its value. The term “inert solvent” when used in the present application is a solvent that does not react with the reactants or reagents under conditions that cause the chemical reaction indicated to take place.

The term “solid dispersion” when used in the present application, denotes a state where most of the Omecamtiv mecarbil or its hydrochloride salt, preferably 90%, 95% or all of Omecamtiv mecarbil or its hydrochloride salt of the solid dispersion, is homogeneously molecularly dispersed in a solid polymer matrix. Preferably solid dispersion, relates to a molecular dispersion where the API (active pharmaceutical ingredient) and polymer molecules are uniformly but irregularly dispersed in a non-ordered way. In other words, in a solid dispersion, the two components (polymer and API) form a homogeneous one-phase system, where the particle size of the API in the solid dispersion is reduced to its molecular size. In a preferred embodiment, in the solid dispersion according to the present invention no chemical bonds can be detected between the API and the polymer. In order to arrive at such a solid dispersion, preferably solid solution, it is required to have a substantial amount of API dissolved in a suitable solvent at least at one time point during preparation of said solid dispersion.

### Examples

### Example-1: Preparation of Amorphous Omecamtiv Mecarbil Dihydrochloride

A solution of Omecamtiv mecarbil dihydrochloride monohydrate (0.250 g) in water (10 mL) was lyophilized to obtain 220 mg of the title compound. PXRD: Amorphous, HPLC purity: 99.90%

### Example-2: Preparation of Amorphous Solid Dispersion of Omecamtiv Mecarbil Dihydrochloride with PVP-K30

A solution of Omecamtiv mecarbil dihydrochloride monohydrate (0.250 g) and PVP K-30 (0.250 g) in water (30 mL) was lyophilized to obtain 460 mg of the title compound. PXRD: Amorphous, HPLC purity: 99.88%

### Example-3: Preparation of Amorphous Solid Dispersion of Omecamtiv Mecarbil Dihydrochloride with Copovidone

A solution of Omecamtiv mecarbil dihydrochloride monohydrate (0.250 g) and Copovidone (0.250 g) in water (30 mL) was lyophilized to obtain 470 mg of the title compound. PXRD: Amorphous, HPLC purity: 99.88%

### Example-4: Preparation of Crystalline Omecamtiv Mecarbil Dihydrochloride Monohydrate Form a

To a solution of Omecamtiv mecarbil dihydrochloride (0.500 g) in water (10 mL), acetonitrile (70 mL) was added at 27° C. and stirred for 3 hours at the same temperature. The solid was filtered and dried under vacuum for 1 hour to obtain 475 mg of title compound. PXRD: Crystalline monohydrate Form A.

### Example-5: Preparation of Premix with Amorphous Omecamtiv Mecarbil Dihydrochloride and Syloid

A solution of Omecamtiv mecarbil dihydrochloride monohydrate (0.250 g) in water (10 mL) was lyophilized to obtain amorphous Omecamtiv mecarbil dihydrochloride. The amorphous product was ground along with Syloid 244 FP (0.250 g) in a mortar and pestle to obtain 450 mg of title premix. PXRD: Crystalline.

### Example-6: Preparation of Premix with Amorphous Solid Dispersion of Omecamtiv Mecarbil Dihydrochloride with PVP-K30 and Syloid

A solution of Omecamtiv mecarbil dihydrochloride monohydrate (0.250 g) and PVP K-30 (0.250 g) in water (15 mL) was lyophilized to obtain amorphous solid dispersion of Omecamtiv mecarbil dihydrochloride with PVP-K30. The amorphous product was ground along with Syloid 244 FP (0.250 g) in a mortar-pestle to obtain 650 mg of the title premix. PXRD: Amorphous.

### Example-7: Preparation of Premix with Amorphous Solid Dispersion of Omecamtiv Mecarbil Dihydrochloride with Co-Povidone and Syloid

A solution of Omecamtiv mecarbil dihydrochloride monohydrate (0.250 g) and co-povidone (0.250 g) in water (15 mL) was lyophilized to obtain amorphous solid dispersion of Omecamtiv mecarbil dihydrochloride with co-povidone. The amorphous product was ground along with Syloid 244 FP (0.250 g) in a mortar and pestle to obtain 660 mg of title premix. PXRD: Amorphous.

### Example-8: Preparation of Premix with Amorphous Solid Dispersion of Omecamtiv Mecarbil Dihydrochloride with Soluplus and Syloid

A solution of Omecamtiv mecarbil dihydrochloride monohydrate (0.250 g) and Soluplus (0.250 g) in water (150 mL) was lyophilized to obtain amorphous solid dispersion of Omecamtiv mecarbil dihydrochloride with Soluplus. The amorphous product was ground along with Syloid 244 FP (0.250 g) in a mortar and pestle to obtain 680 mg of title premix. PXRD: Crystalline.

### Example-9: Preparation of Crystalline Form DP1 of Omecamtiv Mecarbil Dihydrochloride

Omecamtiv mecarbil dihydrochloride (0.5 g) was dissolved in formic acid (15 mL) at 24° C. and the solution was filtered to make it particle free. The solvent from the clear solution was evaporated completely at 60° C. under reduced pressure to obtain 440 mg of the title compound. PXRD: crystalline form DP1

### Example-10: Preparation of Crystalline Form DP2 of Omecamtiv Mecarbil Dihydrochloride

Omecamtiv mecarbil dihydrochloride (1.0 g) was dissolved in acetic acid (15 mL) at 88° C. and the solution was filtered to make it particle free. The clear solution was cooled to 20° C. and stirred the mixture at the same temperature for 1.5 hours. The solid was filtered and dried under vacuum for 2 hours at 40° C. to obtain 0.92 g of the title compound. PXRD: crystalline form DP2.

### Example-11: Preparation of Crystalline Form DP3 of Omecamtiv Mecarbil Dihydrochloride

Crystalline form DP2 of Omecamtiv mecarbil dihydrochloride was dried at 85° C. under vacuum for 1 hour to obtain the title compound. PXRD: crystalline form DP3.

### Example-12: Preparation of Crystalline Form DP4 of Omecamtiv Mecarbil Dihydrochloride

Omecamtiv mecarbil dihydrochloride (0.5 g) was combined with propionic acid (8 mL) at 25° C. and the mixture was heated to 90° C. The mixture was stirred at 90° C. for 16 hours and the solid was filtered. The wet solid was dried under vacuum at 50° C. for 3 hours and at 80° C. for 1 hour to obtain the title compound. PXRD: crystalline form DP4.

### Example-13: Preparation of Crystalline Form OMT-L of Omecamtive Mecarbil Dihydrochloride

Omecamtiv mecarbil dihydrochloride (0.5 g) was combined with DL-lactic acid (5 mL) at 25° C. and the mixture was heated to 50° C. Methyl tert. Butyl ether (30 mL) was added to the mixture at 50° C. and stirred the mixture at the same temperature for 4 hours. The solid was filtered and dried under vacuum at 24° C. for 1 hour and at 50° C. for 5 hours to obtain the title compound. PXRD: crystalline form OMT-L.

### Example-14: Preparation of Crystalline Form OMT-D of Omecamtive Mecarbil Dihydrochloride

Omecamtiv mecarbil dihydrochloride (0.5 g) was dissolved in dimethyl sulfoxide (10 mL) at 24° C. and the solution was filtered to make it particle free. Tetrahydrofuron (30 mL) was added to the solution at 24° C. and stirred the mixture at the same temperature for 2 hours. The solid was filtered and dried under vacuum at 50° C. for 6 hours to obtain the title compound. PXRD: crystalline form OMT-D.

### Example-15: Preparation of Crystalline Form OMT-O of Omecamtive Mecarbil Dihydrochloride

Omecamtiv mecarbil dihydrochloride (0.5 g) and oxalic acid (110 mg) was combined in a mixture of chloroform (10 mL) and water (1 mL) at 24° C. and the mixture was stirred for 16 hours at the same temperature. The solid was filtered and dried under vacuum at 40° C. to obtain the title compound. PXRD: crystalline form OMT-O.

### Example-16: Preparation of Crystalline Form OMT-F of Omecamtive Mecarbil Dihydrochloride and Fumaric Acid

Omecamtiv mecarbil dihydrochloride (1.5 g) and Fumaric acid (403.5 mg) was combined in a mixture of chloroform (15 mL) and water (1.5 mL) at 24° C. and the mixture was stirred for 16 hours at the same temperature. The solid was filtered and dried under vacuum at 26° C. to obtain the title compound. PXRD: crystalline form OMT-F.

### Example-17: Preparation of Crystalline Form OMT-FS of Omecamtiv Mecarbil Dihydrochloride

Omecamtiv mecarbil dihydrochloride (1 g) was dissolved in formamide (10 mL) at 50° C. and the solution was filtered to make it particle free. Tetrahydrofuron (30 mL) was added to the solution at 24° C. and stirred the mixture for 2.5 hours at the same temperature. The solid was filtered and dried under vacuum at 30° C. for 1 hour to obtain the title compound. PXRD: crystalline form OMT-FS.

### Example-18: Preparation of Premix with Amorphous Solid Dispersion of Omecamtiv Mecarbil with Soluplus and Syloid

A solution of Omecamtiv mecarbil dihydrochloride monohydrate (0.250 g) and Soluplus (0.250 g) in a mixture of acetone (40 mL) and water (10 mL) was evaporated in a rotavapor under reduced pressure at 60° C. to obtain amorphous solid dispersion of Omecamtiv mecarbil dihydrochloride with Soluplus. The amorphous solid dispersion (200 mg) was ground along with Syloid AL 1FP (0.100 g) in a mortar and pestle to obtain the title premix. PXRD: amorphous.

### Example-19: Preparation of Premix with Amorphous Solid Dispersion of Omecamtiv Mecarbil with PVP-K30 and Syloid

A solution of Omecamtiv mecarbil dihydrochloride monohydrate (0.250 g) and PVP-K30 (0.250 g) in a mixture of acetone (60 mL) and water (15 mL) was evaporated in a rotavapor under reduced pressure at 60° C. to obtain amorphous solid dispersion of Omecamtiv mecarbil dihydrochloride with PVP-K30. The amorphous solid dispersion (200 mg) was ground along with Syloid AL 1FP (0.100 g) in a mortar and pestle to obtain the title premix. PXRD: amorphous.

### Example-19: Preparation of Premix with Amorphous Solid Dispersion of Omecamtiv Mecarbil with Copovidone and Syloid

A solution of Omecamtiv mecarbil dihydrochloride monohydrate (0.250 g) and Copovidone VA 64 (0.250 g) in a mixture of acetone (90 mL) and water (15 mL) was evaporated in a rotavapor under reduced pressure at 60° C. to obtain amorphous solid dispersion of Omecamtiv mecarbil dihydrochloride with Copovidone. The amorphous solid dispersion (200 mg) was ground along with Syloid AL 1FP (0.100 g) in a mortar and pestle to obtain the title premix. PXRD: amorphous.

### Example-20: Preparation of Crystalline Form OMT-D of Omecamtive Mecarbil Dihydrochloride

Omecamtiv mecarbil dihydrochloride (1 g) was combined with dimethyl sulfoxide (10 mL) at 50° C. and the mixture was stirred overnight at 24° C. The solid was filtered to obtain the title compound. PXRD: crystalline form OMT-D.

1-23. (canceled)

24. A crystalline from of Omecamtiv mecarbil dihydrochloride selected from the group consisting of:
(i) Form DP1 of Omecamtiv mecarbil dihydrochloride, characterized by a PXRD pattern comprising peaks at 6.55, 16.69, 25.53 and 26.42±0.2° 2θ;
(ii) Form DP2 of Omecamtiv mecarbil dihydrochloride, characterized by a PXRD pattern comprising peaks at 5.89, 17.94, 25.58 and 30.88±0.2° 2θ;
(ii) Form DP3 of Omecamtiv mecarbil dihydrochloride, characterized by a PXRD pattern comprising peaks at 25.64, 13.93 and 31.17±0.2° 2θ;
(iv) Form DP4 of Omecamtiv mecarbil dihydrochloride, characterized by a PXRD pattern comprising peaks at 6.8, 12.62 and 18.54±0.2° 2θ;
(v) Form OMT-L of Omecamtiv mecarbil dihydrochloride and Lactic acid, characterized by a PXRD pattern comprising peaks at about 5.87, 17.84, 23.8, 25.35 and 27.91±0.2° 2θ;
(vi) Form OMT-D of Omecamtiv mecarbil dihydrochloride, characterized by a PXRD pattern comprising peaks at 12.98, 15.79, 18.55 and 25.63±0.2° 2θ;
(vii) Form OMT-O of Omecamtiv mecarbil dihydrochloride and Oxalic acid, characterized by a PXRD pattern comprising peaks at about 15.69, 24.26 and 29.44±0.2° 2θ;
(viii) Form OMT-F of Omecamtiv mecarbil dihydrochloride and fumaric acid, characterized by a PXRD pattern comprising peaks at about 6.14 and 17.37°±0.2° 2θ;
(ix) Form OMT-FS of Omecamtiv mecarbil dihydrochloride characterized by a PXRD pattern comprising peaks at about 24.68, 25.61 and 31.06°±0.2° 2θ; and combinations thereof.

25. A pharmaceutical composition comprising one or more of the crystalline forms of Omecamtiv mecarbil dihydrochloride defined in claim 24 and at least one pharmaceutical acceptable excipient.

26. A process for the preparation of the crystalline forms of Omecamtiv mecarbil dihydrochloride defined in claim 24, comprising a step of contacting the solution of Omecamtiv mecarbil dihydrochloride in a solvent with an anti-solvent.

27. The process of claim 26, wherein the solvent is selected from the group consisting of water, dimethyl sulfoxide, dimethylformamide, N-methylpyrrolidone, methanol, ethanol, 2-propanol, dichloromethane and mixtures thereof; and the anti-solvent is selected from the group consisting of acetonitrile, methyl acetate, ethyl acetate, isopropyl acetate, acetone, methyl ethyl ketone, methyl isobutyl ketone, tetrahydrofuran, 1,4-dioxane, hexane and mixtures thereof.

28. A process for the preparation of crystalline form DP1 of Omecamtiv mecarbil dihydrochloride as defined in claim 24, comprising the step of contacting Omecamtiv mecarbil dihydrochloride with formic acid.

29. A process for the preparation of crystalline form DP2 of Omecamtiv mecarbil dihydrochloride as defined in claim 24, comprising the step of contacting Omecamtiv mecarbil dihydrochloride with acetic acid.

30. A process for the preparation of crystalline form DP3 of Omecamtiv mecarbil dihydrochloride as defined in claim 24, comprising the step of heating crystalline form DP2 of Omecamtiv mecarbil dihydrochloride.

31. A process for the preparation of crystalline form DP4 of Omecamtiv mecarbil dihydrochloride as defined in claim 24, comprising the steps of:
(a) contacting Omecamtiv mecarbil dihydrochloride with propionic acid;
(b) isolating Omecamtiv mecarbil dihydrochloride; and
(c) optionally, heating crystalline Omecamtiv mecarbil dihydrochloride of step (b).

32. A process for the preparation of crystalline form OMT-L of Omecamtiv mecarbil dihydrochloride as defined in claim 24, comprising the steps of:
(a) contacting Omecamtiv mecarbil dihydrochloride with DL-Lactic acid;
(b) isolating Omecamtiv mecarbil dihydrochloride; and
(c) optionally, heating crystalline Omecamtiv mecarbil dihydrochloride of step b).

33. A process for the preparation of crystalline form OMT-D of Omecamtiv mecarbil dihydrochloride as defined in claim 24, comprising the steps of:
(a) contacting Omecamtiv mecarbil dihydrochloride with dimethyl sulfoxide;
(b) isolating Omecamtiv mecarbil dihydrochloride; and
(c) optionally, heating Omecamtiv mecarbil dihydrochloride of step (b).

34. A process for the preparation of crystalline form OMT-O of Omecamtiv mecarbil dihydrochloride and Oxalic acid as defined in claim 24, comprising the step of contacting Omecamtiv mecarbil dihydrochloride with Oxalic acid.

35. A process for the preparation of crystalline form OMT-F of Omecamtiv mecarbil dihydrochloride and Fumaric acid as defined in claim 24, comprising the step of contacting Omecamtiv mecarbil dihydrochloride with Fumaric acid.

36. A process for the preparation of crystalline form OMT-FS of Omecamtiv mecarbil dihydrochloride as defined in claim 24, comprising the step of contacting Omecamtiv mecarbil dihydrochloride with formamide.

37. An amorphous solid dispersion of Omecamtiv mecarbil dihydrochloride together with at least one pharmaceutically acceptable excipient.

38. A premix comprising the amorphous solid dispersion of claim 37 and a silicon dioxide.

39. A process for the preparation of amorphous solid dispersion of Omecamtiv mecarbil dihydrochloride as defined in claim 37, comprising the steps of providing a solution of Omecamtiv mecarbil dihydrochloride together with at least one pharmaceutically acceptable excipient in a solvent and removing the solvent.

40. A pharmaceutical composition comprising the amorphous solid dispersion of Omecamtiv mecarbil dihydrochloride as defined in claim 37 and one or more of the crystalline forms of Omecamtiv mecarbil dihydrochloride defined in claim 24.
